(19) United States
(12) Patent Application Publication
(10) Pub. No.: US 2009/0060935 A1
(43) Pub. Date: Mar. 5, 2009

(54) AMEBIASIS VACCINE

(76) Inventors: Hiroshi Tachibana, Kanagawa (JP); Xunjia Cheng, Kanagawa (JP)

Correspondence Address: HOVEY WILLIAMS LLP, 10801 Mastin Blvd., Suite 1000, Overland Park, KS, 66210, US

(21) Appl. No.: 12/177,994

(22) Filed: Jul. 23, 2008

(30) Foreign Application Priority Data
Jul. 23, 2007 (JP) 2007-190535

(51) Int. Cl. A61K 39/002; C07K 14/00; C12N 15/30; C12N 15/74; A61P 33/04; C07K 16/44; C12N 1/00; C12P 21/02

(52) U.S. Cl. 424/191.1; 530/350; 536/23.1; 435/320.1; 435/243; 435/69.3; 530/387.9

(57) ABSTRACT

An isolated protein fragment containing an epitope which is specific for E. histolytica and is commonly shared by all polymorphic strains of E. histolytica is provided, and which exhibits immunogenicity in the host, and comprises an amino acid sequence containing amino acid sequence 603-1088 of SEQ ID NO: 2, and in particular, consists of the amino acid sequence 603-1088. An isolated DNA coding for such fragment, a vector containing such DNA, a host cell, a vaccine for amebiasis containing such fragment having a low molecular weight which can be stably produced in the host cell or E. coli in a large amount and at a high production efficiency, a method for producing such vaccine, an antibody against such fragment, and a method for preventing or treating amebiasis by administering such fragment are also provided.

This application claims priority on Japanese Patent Application No. 2007-190535 which was filed on Jul. 23, 2007, the entire content of which are hereby incorporated by reference. In addition, the entire content of literatures cited in this specification are incorporated by reference.

### SEQUENCE LISTING

A printed Sequence Listing accompanies this application, and has also been electronically submitted with identical contents in a computer-readable ASCII file.

### BACKGROUND OF THE INVENTION

This invention relates to a protein fragment including epitope region of an intermediate subunit of a surface adhesion factor of Entamoeba. histolytica. This invention also relates to a vaccine for preventing or treating the infection caused by E. histolytica wherein the vaccine contains the protein fragment as an immunogen.

Of the world population, about 500 million people are infected by the protozoan parasite (Entamoeba histolytica/E. dispar) which had been conventionally known as E. histolytica, and about 10% of such population develop dysentery, colitis, or liver abscess by the pathogenic E. histolytica (Entamoeba histolytica), resulting in the annual death toll of 40,000 to 110,000. This death toll is the third largest among the parasitic diseases, next to malaria and schistosomiasis. The infection is distributed throughout the world, but especially in the tropical and subtropical regions, and in Japan where the number of domestic infection exceeds the number of imported case, the reported number of the amebiasis treated as a Type V infection is increasing year after year. Unlike Europe or the US, amebiasis is increasing among male homosexual population, and mixed infection with HIV is also frequent in Japan. The number reported in 2006 in IDWR of Ministry of Health, Labour and Welfare/Infectious Disease Surveillance Center was 738. This number is the third among the infections with total number report after enterohemorrhagic Escherichia coli infection and acquired immunodeficiency syndrome.

Symptom of the amebiasis is known to be exacerbated by the administration of adrenocorticosteroid or pregnancy. Metronidazole which is widely used as a medication for amebiasis is counterindicated for pregnant women. Development of a vaccine is highly awaited especially in view of the fact that the central region of the amebiasis is tropical and subtropical regions.

Since the process of adhesion to the host cell is essential for the pathogenicity of E. histolytica, surface lectin which is a protein having a sugar chain-specific binding ability has been studied as an important protein candidate for the vaccine (see, for example, PETRI, W. A., JR., HAQUE, R. and MANN, B. J., “The bittersweet interface of parasite and host: lectin-carbohydrate interactions during human invasion by the parasite Entamoeba histolytica,” Annu Rev Microbiol, 2002, 56, pp. 39-64 (Non-patent Document 1)). For example, in WO97/18790 (Patent Document 1), use of a C-type lectin for the vaccine for microorganisms such as parasites is proposed, and in this document, E. histolytica is disclosed as an example of the parasite.

In specific relation to the E. histolytica, the most studied lectin is a surface lectin of 260 kDa which is specific for galactose (Gal) and N-acetylgalactosamine (GalNAc). This Gal/GalNAc adhesion lectin was isolated from HM-1:IMSS strain of E. histolytica, and has been known to be a molecule comprising a heavy subunit of 170 kDa (heavy subunit: HGL) and a light subunit of 35/31 kDa (light subunit: LGL) connected by S—S bond. Of these two subunits, the site which recognizes the sugar chain is located on the HGL (see, for example, PETRI, W. A., JR., CHAPMAN, M. D., SNODGRASS, T., MANN, B. J., BROMAN; J. and RAVDIN, J. I., “Subunit structure of the galactose and N-acetyl-D-galactosamine-inhibitable adherence lectin of Entamoeba histolytica”, J. Biol Chem, 1989, 264, pp. 3007-3012. (Non-patent Document 2).

With regard to the use of this HGL for the vaccine, for example, WO95/00849 (Patent Document 2) proposes a vaccine including, an epitope-bearing region in the HGL of E. histolytica, which is produced as recombinant protein in prokaryotic cell culture system and thus is not glycosylated. Of the amino acid sequence constituting the HGL moiety, three amino acid regions (596-818, 1033-1082, and 1082-1138) are disclosed as the pathotype-specific epitopes included in the 170 kDa protein of the HGL moiety.

Animal experiments have been conducted for several proteins as being amebiasis vaccine candidates. However, no vaccine is yet in the actual use (CHAUDHRY, O. A. and PETRI, W. A., JR. “Vaccine prospects for amebiasis, Expert Rev Vaccines,” 2005, 4, pp. 657-668 (Non-patent Document 3)). In addition, E. histolytica is known to have polymorphism, and there is a demand for a vaccine which is effective for all strains.

### SUMMARY OF THE INVENTION

An object of the present invention is to provide a protein fragment which is useful as an effective vaccine for all types of E. histolytica; and in particular, to provide a low molecular weight protein fragment of the epitope region, its recombinant fragment, and an amebiasis vaccine containing such recombinant fragment which can be stably supplied in a large amount.

In the course of investigating E. histolytica, the inventors of the present invention succeeded in selecting a monoclonal antibody which exhibits specificity for E. histolytica (hereinafter referred to as EH3015), and found that this antibody EH3015 has the an in vitro action of inhibiting cell adhesion as well as inhibitory action on the formation of liver abscess by E. histolytica in hamster. The inventors also found that the protein fragment identified by the antibody EH3015 is a molecule having a molecular mass of 150 kDa which has some association with the heavy chain HGL and light chain IGL of Gal/GalNAc adhesion lectin but clearly different from these molecules. Since this protein fragment is intermediate subunit of a surface adhesion factor related to HGL and LGL, it is referred to as “IGL”, and this protein fragment is also referred “IGL” in this specification.

For IGL, presence of two genes, namely, a gene coding for 1101 amino acid residues (hereinafter referred to as IGL1) and a gene coding for 1105 amino acid residues (hereinafter referred to as IGL2) has been confirmed by gene cloning of HM-1:IMSS (the standard strain of E. histolytica). The inventors of the present invention studied expression of these two isotypes IGL1 and IGL2, and found that the amount of the IGL1 expressed was significantly higher than that of the IGL2 in both of the pathogenic E. histolytica and the non-pathogenic E. dispar. The inventors also found that, while the amount of the IGL2 expression is not different between E. histolytica and E. dispar, amount of the IGL1 expression was higher in E. histolytica compared to E. dispar. This suggests that IGL1 has stronger association with the pathogenicity compared to IGL2.

In the immunization test in hamster using a recombinant protein of IGL1 prepared in E. coli, it was found that protective immunity for the formation of liver abscess by E. histolytica can be provided with the animal, and effectiveness of this recombinant protein for use as a vaccine was thereby confirmed. Furthermore, in the test using the recombinant fragment of IGL1, the antigen epitope(s) of IGL 1 was found to be included in the fragment on the C terminal side (C-IGL). It was also confirmed that the two isotypes IGL1 and IGL2 of the IGL have little difference in their primary structure on the C terminal side, and no remarkable difference in the C terminal side is found among the polymorphic IGLs.

Based on these findings, the immunological effects by the fragment including the epitope region of IGL1 from the HM-1:IMSS strain is presumably not merely effective for particular strains but all E. histolytica strains distributed throughout the world. And the vaccine including such fragment as the immunogen can be stably supplied at a large amount. Accordingly, the present invention as described below is provided.

The present invention provides an immunogenic E. histolytica protein fragment, and in particular, an isolated protein fragment including amino acid sequence 603-1088 of SEQ ID NO: 2. More specifically, the present invention provides an isolated protein fragment comprising amino acid sequence 603-1088 of SEQ ID NO: 2; or an isolated protein fragment comprising amino acid sequence 14-1088 of SEQ ID NO: 2 including such region. These protein fragments may include those with deletion, substitution, or addition of one to several amino acid residues in such amino acid sequence as long as the fragment has an immunogenicity equivalent to the isolated protein fragment.

The amino acid sequence represented by the SEQ ID NO: 2 is the full length amino acid sequence of IGL1 from E. histolytica HM-1:IMSS strain (Deposit No. ATCC30459), and it corresponds to the nucleotide sequence 1-3303 of SEQ ID NO: 1 which is a nucleotide sequence comprising 3306 nucleotides at 15957-19262 of the locus 53.m00171 in whole genome of the HM-1:IMSS strain.

SEQ ID NO: 1 is the nucleotide sequence corresponding to IGL1. In other words, this sequence codes for the fragment including the epitope region in the intermediate subunit of the surface adhesion factor of E. histolytica.

The protein fragment comprising amino acid sequence 603-1088 of SEQ ID NO: 2 (hereinafter sometimes referred to as C-IGL) is a fragment on the side of the C terminal comprising 486 amino acid residues including the epitope region of the IGL1, and the protein fragment of 1075 amino acid residues comprising the amino acid sequence 14-1088 (hereinafter sometimes referred to as F-IGL) is a full length fragment of IGL1 excluding the signal sequence on the N terminal and the C terminal.

The protein fragment as described above is free from the sugar chain, and the protein fragment is preferably a recombinant fragment produced in prokaryotes which is typically E. coli. Of the protein fragments including the epitope region, the preferred is the recombinant fragment of the protein fragment C-IGL comprising the short amino acid sequence of 603-1088 which exhibits high production efficiency. The recombinant fragment may be produced by the genetic engineering method commonly used in the art comprising the steps of preparing an expression vector including the DNA coding for the protein fragment, transforming the host cell with the vector, and collecting the thus produced protein fragment. This present invention also provides such method for producing the protein fragment, and a vector including the DNA coding for such protein fragment.

This protein fragment is effective for treating and preventing the infection caused by amebiasis. Accordingly, the present invention is capable of providing an amebiasis vaccine containing such protein fragment as a component which exhibits immunogenicity in the host. This vaccine may be a composition containing an adjuvant together with the protein fragment.

The protein fragment of the present invention contains an epitope which is specific for E. histolytica, and which is commonly shared by all polymorphic strains of E. histolytica, and this protein fragment is also immunogenic. The administration of such protein fragment is vaccine effective for amebiasis, and this effect is realized by produced antibodies inhibiting infection process through cell adhesion of E. histolytica or damaging E. histolytica through a complement- or ADCC-mediated action, or by immunological mechanism such as cell-mediated immunity. Since the protein fragment of the present invention is a small molecule, it will realize an amebiasis vaccine which can be stably mass-produced with a good production efficiency in E. coli system.

### BRIEF DESCRIPTION OF THE DRAWINGS

FIG. 1 is a graph showing amount of the IGL1 and IGL2 genes expressed by real time RT-PCR in relative ratio.

FIG. 2 shows electropherograms of the recombinant IGL1 in polyacrylamide gel electrophoresis.

FIG. 3 shows images of the liver of the hamster, which had been immunized with the recombinant full length IGL, at 7 days after the inoculation of E. histolytica trophozoites.

FIG. 4 shows images of the liver of the hamster, which had been immunized with the recombinant IGL fragment, at 7 days after the inoculation of E. histolytica trophozoites.

### DETAILED DESCRIPTION OF THE PREFERRED EMBODIMENT

The present invention provides an immunogenic protein fragment found by the inventors of the present invention. This fragment includes the epitope region of the intermediate subunit of the surface adhesion factor from HM-1:IMSS strain of E. histolytica. More specifically, this fragment is based on IGL1 of the two protein fragments IGL1 (1101 amino acid) and IGL2 (1105 amino acid) whose presence has been confirmed by cloning of the gene in HM-1:IMSS strain for the protein fragment IGL identified as described above by the mouse monoclonal antibody EH3015.

The nucleotide sequence of the gene coding for the IGL1 of HM-1:IMSS strain used in the present invention is shown in SEQ ID NO: 1, and the amino acid sequence corresponding to the nucleotide sequence is shown in SEQ ID NO: 2.

The HM-1:IMSS strain of E. histolytica has been on deposit of ATCC30459. The genome of the HM-1:IMSS strain has been analyzed (http://www.tigr.org/tdb/e2k1/eha1/), and the genes coding for the protein fragments IGL1 and IGL2 as described above have been described in the article of the inventors of the present invention (“Intermediate subunit of the Gal/GalNAc lectin of Entamoeba histolytica is a member of a gene family containing multiple CXXC sequence motifs “Infect Immun (20011), 69, 5892-5898). This article also describes absence of substantial difference in the primary structure of the C terminal side between the IGL1 and the IGL2, and this article is incorporated herein by reference in its entirety.

It is to be noted that the primary structure of the IGL1 is clearly different from that of the HGL and LGL which have been already investigated. More specifically, no significant similarity was found between the amino acid sequence of IGL1 (SEQ ID NO: 2) and the amino acid sequence of HGL in the comparison conducted by using Blast2 sequence program. In addition, 15957-19262 of locus 53.m00171 of IGL1 in the E. histolytica (total genome in HM-1:IMSS strain) is different from 33710-37570 of locus 16.m00300 and 32859-36733 of locus 29.m00206 in the HGL.

When these two isotypes of the IGL were compared for their amount of expression in E. histolytica (HM-1:IMSS) by the real time RT-PCR test as described below, the amount of the IGL1 gene expressed was found to be 6.4 times (relative ratio) larger than that of the IGL2 gene. In the comparison between E. histolytica and non-pathogenic E. dispar, no difference was found for the amount of the IGL2 gene expressed while the amount of the IGL1 gene expressed was higher in E. histolytica compared to the E. dispar. These results indicate higher association of the IGL1 with the pathogenicity.

Presence of polymorphism in the primary structure of IGL has also been found in the evaluation of E. histolytica strains isolated from various regions in the world.

Homology of the full length amino acid sequence of the IGL1 between the polymorphic strains has been reported, for example, for HM-1:IMSS, DKB, HK-9, HB-301:NIH, NOT-12, YS, and YI in IASR, April 2007, vol. 28, pp. 110 to 111, which is hereby incorporated by reference in its entirety. As described in this article, a homology as high as 99.9% has been found for the full length amino acid sequence of IGL1 between the standard strain HM-1:IMSS and DKB strain (from England).

The sequence was completely the same for HK-9 strain (from Korea) and HB-301:NIH strain (from Myanmar), which were both isolated in Asia, and the homology between these strains and the standard strain HM-1:IMSS was 86%. The sequence was also completely the same for NOT-12, YS, and YI strains which were isolated in Japan, and the homology between these strains and the standard strain HM-1:IMSS was 88%.

Further study conducted by the inventors of the present invention confirmed that the polymorphism is greater particularly on the side of the N terminal, and the C terminal side is substantially consistent for various strains. Accordingly, of the latter two groups as mentioned above, F-IGL had lower homology to the HM-L:IMSS than DKB strain, whereas C-IGL exhibited high homology.

More illustratively, genes respectively coding for the N terminal side, C terminal side, and middle region of the IGL1 were amplified by PCR, and these gene fragments were incorporated in an expression vector to produce the three fragments each having the amino acid sequence as shown in Table 2 of the Example as described below.

For the amino acid sequence of C-IGL, homology of the HK-9 strain to the HM-1:IMSS strain was 94%, and the homology of the NOT-12 strain to the HM-1:IMSS strain was 97%. Based on these findings, the immunological effects as will be apparent from the Examples as described below by the epitope region from the IGL1 of the HM-1:IMSS strain is not merely effective for particular strains but has a high probability of exhibiting the vaccine effect for any E. histolytica strains throughout the world wide distribution.

Since C-IGL comprising 486 amino acid residues which has a size smaller than the full length F-IGL, C-IGL can be readily expressed in E. coli at a sufficient amount, can produce much more product. C-IGL can be collected in the form of inclusion body, and a highly pure recombinant C-IGL can be obtained in 3 to 4 washing steps.

HGL has three isotypes. IGL which has two isotypes presumably has a higher vaccine effect per one type of the recombinant protein than the HGL. IGL is also more cysteine-rich than HGL, and presumably, has a firm steric structure. It is thus presumed that the epitope formed by such steric structure is present in IGL.

While IGL has no site for sugar chain recognition, IGL has been confirmed to be a surface adhesion factor in the Examples as described below. The cell adhesion mechanism of the IGL is not clearly analyzed. However, since behavior of the IGL is closely related to HGL and LGL of Gal/GalNAc specific lectin, surface adhesion of the IGL is presumably realized by bonding of IGL with HGL-LGL dimer having a sugar chain-recognition site by non-covalent bonding which results in the formation of a complex. Recently, for IGL, it has been reported to have a binding ability for villi of the small intestine (Cellular Microbiology, 2005, 7, 569-579), and IGL2 has also been reported to have a binding ability for fibronectin (Parasitology, 2007, 134, 169-177).

The antibody EH3015 used for the identification of the IGL is a mouse monoclonal antibody whose specificity for E. histolytica had been identified in the course of establishing the method for differentiating the pathogenic E. histolytica from the non-pathogenic E. dispar by using the mouse monoclonal antibody among those morphologically identified as E. histolytica/E. dispar. The inventors of the present invention have reported that adhesion to erythrocyte and Chinese hamster ovary (CHO) cell, ability of erythrophagocytosis, and cytotoxicity for CHO could be prevented in vitro by pretreating E. histolytica with antibody EH3015 (“A monoclonal antibody against the 150 kDa surface antigen of Entamoeba histolytica inhibits adherence and cytotoxicity to mammalian cells” Med Sci Res 1997), 25, 159-161), and that formation of the liver abscess could be suppressed at a significant level when EH3015 was abdominally administered in hamster and E. histolytica was directly inoculated into the liver at 24 hours after the administration (“Protection of hamsters from amebic liver abscess formation by a monoclonal antibody to a 150 kDa surface lectin of Entamoeba histolytica” Parasitol Res (1999), 85, 78-80).

The inventors also reported that the liver abscess formation could be suppressed when the hamster was immunized by using a protein purified from E. histolytica in an EH3015 affinity column. (“Protection of hamsters from amebic liver abscess formation by immunization with the 150 kDa and 170 kDa surface antigens of Entamoeba histolytica” Parasitol Res (2001), 87, 126-130).

However, production of a large amount of IGL by such method has been difficult. In addition, the effect of other proteins which are present in a trace amount can not be denied.

On the other hand, it is yet unknown whether protein IGL identified by EH3015, and in particular, the recombinant fragment of the IGL1 from HM-1:IMSS strain produced in E. coli which is free from the sugar chain is immunologically effective as in the case of the protein purified from E. histolytica as described above. In order to determine the possibility of using the IGL as a vaccine which can be stably supplied at a large amount, the recombinant IGL1 produced in E. coli was used to test its immunity for capability of preventing the amebic liver abscess. The result was favorable as described in the section of the Examples.

With regard to the IGL1, favorable result for the vaccine effect was obtained for the antigen on the C terminal side, as reported in “Evaluation of recombinant fragments of Entamoeba histolytica Gal/GalNAc lectin intermediate subunit for serodiagnosis of amebiasis” J Clin Microbiol(2004), 42, 1069-1074, in the evaluation of the full length or fragment recombinant protein that has been prepared in E. coli as an antigen for serum diagnosis.

In the infection by E. histolytica, cyst becomes trophozoite in small intestine, and this trophozoite adheres to large intestinal mucosa, and invades into the tissue to form ulcer, thereby causing colitis. When the trophozoite metastasizes hematogenously from the intestinal tissue to liver, liver abscess is formed, and this liver abscess is fatal when left untreated. In view of the immunoreaction of the amebiasis, vaccination using the protein fragment of the present invention as the immunogenic component is highly likely to be effective in treating and preventing the infection.

The vaccine of the present invention may by prepared by further adding a compound which is commonly used as an adjuvant in the vaccine composition.

### EXAMPLES

Next, the present invention is described in further detail by referring to the following Examples which by no means limit the scope of the present invention.

HM-1:IMSS strain (E. histolytica), CYNO9 strain (E. dispar), and SAW1734 strain (E. dispar) were obtained from Department of Tropical Medicine and Parasitology, Keio University School of Medicine.

### Example 1

### Real Time RT-PCR Analysis of the Amount of IGL Gene Expressed

Expression of the IGL1 and IGL2 genes of the E. histolytica was compared by real time RT (reverse transcription)-PCR.

Full length RNA was isolated from the trophozoite cultures of E. histolytica and E. dispar by using RNeasy mini-kit (Qiagen), and cDNA was synthesized by using GeneAmp RNA PCR kit (Applied Biosystems).

The reaction mixture for use in the quantitative real time RT-PCR analysis was prepared by mixing SYBR Premix Ex Taq (Takara Shuzo Co., Ltd.), specific primer as described below, Rox dye, and the cDNA.

### [Table 1]

TABLE 1

Gene

| | | |
|---|---|---|
| Type | Region | Primer |

| | | | |
|---|---|---|---|
| E. dispar | IGL1 | forward | 5′-TGA CAA AGA CAA |
| | | | TAC TTG TAA AAA |
| | | | GTG-3′ |
| | | reverse | 5′-ATT ACT AAC ACA |
| | | | TGC ACA TTT TTT |
| | | | GTC-3′ |
| | IGL2 | forward | 5′-TCG ATG AAA ATA |
| | | | ATG TAT GCC AGA |
| | | | AAT-3′ |
| | | reverse | 5′-TCA TCA AGG CAA |
| | | | GCA CAT TGA CTA-3′ |
| E. histolytica | IGL1 | forward | 5′-GTT CAC AGG TTG |
| | | | GTG CTT GTA CG-3′ |
| | | reverse | 5′-ACA GTA CAT GGC |
| | | | TTT TCT CCG GTA-3′ |
| | IGL2 | forward | 5′-GAT TCA CAA ACA |
| | | | AAG GAG TGT GCC-3′ |
| | | reverse | 5′-GTG CAT TTG AAC |
| | | | CAC TAG CAG CAA-3′ |
| Actin | | forward | 5′-CCA GCT ATG TAT |
| | | | GTT GGA ATT CAA G-3′ |
| | | reverse | 5′-GAT CAA GTC TAA |
| | | | GAA TAG CAT GTG G-3′ |

Amplification was conducted for 40 cycles, and in each cycle, fluorescence intensity was measured by a sequence detection system ABI PRISM 7700 (Applied Biosystems, software ver. 1.7).

The experiment was conducted by shuttle PCR protocol comprising initial denaturing at 95° C. for 10 seconds, denaturing at 95° C. for 5 seconds, and annealing/extension at 60° C. for 30 seconds.

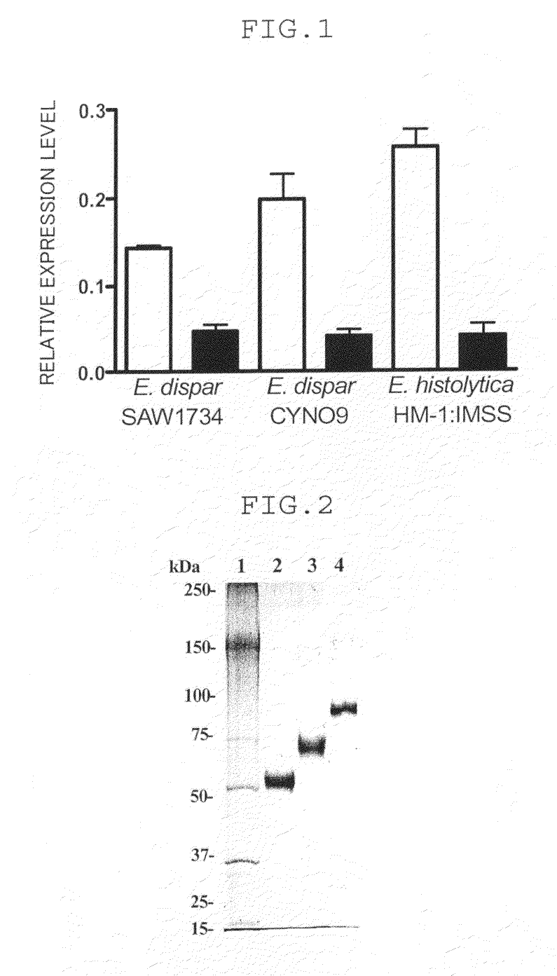

Relative expression level on each gene of IGL1 and IGL2 was analyzed by comparative CT method using an actin gene for the control (internal control). The experiment was conducted three times including the cultivation step and the RNA isolation step to thereby calculate average and standard deviation. Expression of each gene is shown in FIG. 1 as a relative value in relation to the expression of the actin gene. In FIG. 1, IGL1 is shown by the blank bar, and IGL2 is shown by solid bar.

As shown in FIG. 1, IGL1 was expressed in E. histolytica at an amount about 6.4 times higher than the IGL2, namely, at a level significantly higher than the IGL2. IGL1 was also expressed in E. dispar at an amount about 3 to 5 times higher than the IGL2, namely, at a level significantly higher than the IGL2. No difference was observed in the expression of the IGL2 between the E. histolytica and the E. dispar, whereas IGL1 was expressed at a higher level in E. histolytica. These results indicate that the IGL1 may have a stronger association with the pathogenicity compared to the IGL2.

### Example 2

### Preparation of Recombinant Protein

The recombinant IGL protein was prepared basically on the bases of the method disclosed in TACHIBANA, H., CHENG, X. J., MASUDA, G., HORIKI, N. and TAKEUCHI, T. (2004), Evaluation of recombinant fragments of Entamoeba histolytica Gal/GalNAc lectin intermediate subunit for serodiagnosis of amebiasis. J Clin Microbiol, 42, 1069-1074.

A gene fragment coding for the full length amino acid excluding the signal sequences at the N and C termini from E. histolytica IGL1 for (SEQ ID NO: 1) [F-IGL, amino acid No. (aa) 14-1088] was amplified by PCR.

The fragment was then expressed by incorporating in XhoI site of pET19b vector, and introducing the vector in E. coli BL21 Star™ (DE3) pLysS.

E. coli was centrifuged, fractured by ultrasonication in a surfactant solution, and centrifuged to obtain the precipitate. This procedure was repeated 3 to 4 times. This washing procedure was conducted by the protocol indicated in the Protein refolding kit (Novagen). The resulting inclusion body was refolded by the method indicated in Protein refolding kit, and purified by His-bound resin affinity chromatography to obtain full length recombinant protein F-IGL.

Similarly, gene fragments respectively coding for N terminal side (N-IGL, aa 14-382), middle region (M-IGL, aa 294-753), and C terminal side (C-IGL, aa 603-1088) of the IGL1 were amplified by PCR, and these fragments were incorporated in XhoI site of the pET19b vector. The vector was introduced into E. coli BL21 Star™ (DE3) pLysS for expression.

Inclusion body was obtained by the washing procedure as described above. The inclusion body was refolded to obtain the recombinant proteins N-IGL, M-IGL, and C-IGL. The recombinant IGL fragment had the purity high enough for determination as a single band in electrophoresis with no further purification by affinity chromatography. These recombinant IGLs are shown in Table 2.

### [Table 2]

TABLE 2

| | | Amino acid sequence |
|---|---|---|
| Recombinant protein | Region | (aa) |
| F-IGL | Full length | 14-1088 |
| N-IGL | Region on the side | 14-382 |
| | of N terminal | |
| M-IGL | Middle region | 294-753 |
| C-IGL | Region on the side | 603-1088 |
| | of C terminal | |

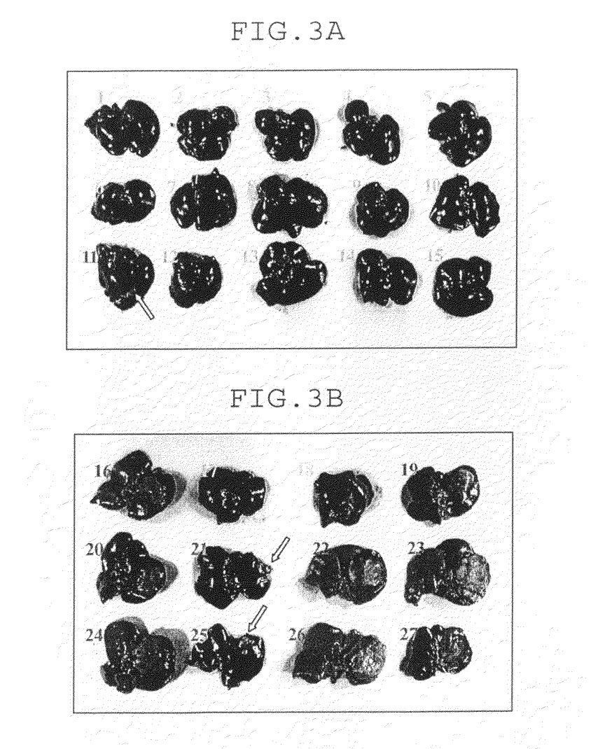

The resulting each recombinant IGL was subjected to electrophoresis. In each lane, 4 μg of the refolded protein was electrophoresed under reducing conditions using 7.5% gel (polyacrylamide gel), and the protein band was stained with Coomassie brilliant blue. The thus obtained electropherograms are shown in FIG. 2.

In FIG. 2, lane 1 is F-IGL, lane 2 is N-IGL, lane 3 is M-IGL, and lane 4 is C-IGL. The numbers on the left hand side indicate molecular mass (unit, kDa) of the protein marker.

### Test Example 1

### Experiment on Immunization by Full Length Recombinant IGL1 and Infection

In the following animal experiment, E. histolytica SAW755CR strain (obtained through Keio University) retaining relatively high infectivity was used instead of the HM-1:IMSS strain which has become less infective to hamster by acclimatization. SAW755CR is a strain isolated in the U.K. in 1979 by Sargeaunt et al. from an Egyptian patient suffering from amebic colitis, and primary structures of the SAW755CR strain and the HM-1:IMSS strain are substantially the same for the full length, and homology of the IGL1 for these strains are 99.9%.

Twenty-seven male Syrian hamsters having a body weight of 40 to 50 g were used. A small amount of blood was collected from ophthalmic vein before the immunization.

In this test, 50 μg of F-IGL was intramuscularly inoculated at the hind leg of the animal with TiterMax Gold (adjuvant). At 3 weeks and 5 weeks after the initial sensitization, the animal was boostered with the same amount of F-IGL and Freund's incomplete adjuvant.

Blood was collected at 1 week after the final sensitization, and after 1 week, 5×1 trophozoites of E. histolytica SAW755CR strain was inoculated into the liver. After 1 week, the liver was checked for the formation of the liver abscess, and total weight of the liver and the weight of the abscess were measured.

The control group was sensitized only by phosphate buffered saline (PBS) and the adjuvant, and the experiment was conducted by the same procedure.

Inhibitory effect of intramuscular inoculation of the full length recombinant IGL (F-IGL) of E. histolytica.for the formation of amebic liver abscess in hamster are shown in Table 3 and FIG. 3.

In FIG. 3, (A) is the group sensitized with F-IGL (1 to 15), and (B) is the control group inoculated with PBS (16 to 27).

In the group sensitized with F-IGL, small abscess (indicated by the arrow) was found only in 11.

In the control group inoculated with PBS, no abscess was found in 17 and 18, and small abscess (indicated by the arrow) was found in 21 and 25. However, formation of a large abscess was found in other 8 animals.

### [Table 3]

TABLE 3

| | | | Inhibition of | Average |
|---|---|---|---|---|
| | | Number of | the abscess | abscess |
| | Immunization | animals with | formation | size |
| Group | antigen | abscess | (%) | (wt %) |
| Control | PBS | 10/12 | 17 | 31 |
| 1 | F-IGL | 1/15* | 93 | 3 |

In relation to the control, P = 0.0000012

As shown in Table 3, in the case of the control using the PBS, the abscess was found in 10 out of 12 animals (83%), while the abscess was found only in 1 out of 15 animals (7%) in the case of the group immunized with F-IGL. The difference was significant (P=0.0000012) in Fisher's test (Fisher's exact probability test).

Average size of the abscess in the hamsters having the abscess was 31% (weight ratio) in the control group, whereas the size of the abscess in the liver was 3% in the one animal having the abscess of the F-IGL group.

### Test Example 2

### Inhibitory Activity for Adhesion of E. histolytica to CHO Cell in an in Vitro System

Pooled sera of the hamsters after the immunization by the F-IGL were diluted to various concentration, and brought in contact with the trophozoites of the HM-L:IMSS strain of E. histolytica on ice for 1 hour. After washing with PBS, the reaction with the CHO cells was conducted on ice for 2 hours. Percentage of the E. histolytica trophozoites having 3 or more CHO cells adhered thereto was measured.

Pooled sera before the immunization were used for the control.

The inhibitory effect of the serum of the immunized hamster for the adhesion between E. histolytica and CHO cell is shown in Table 4.

As shown in Table 4, the serum immunized with F-IGL suppressed adhesion of the trophozoites to the CHO cell to the level of 27% compared to the control group at the 10 fold dilution, and to the level of 40% at the 100 fold dilution.

### [Table 4]

TABLE 4

| Group | Immunization antigen | Diluted serum | Adhesion(%)* |
|---|---|---|---|
| Control | PBS | 1:10 | 99 |
| Control | PBS | 1:100 | 111 |
| Control | PBS | 1:1000 | 101 |
| 1 | F-IGL | 1:10 | 27 |
| 1 | F-IGL | 1:100 | 40 |
| 1 | F-IGL | 1:1000 | 98 |

*Comparison with the serum before the immunization

As shown above, it has been for the first time demonstrated that the full length recombinant protein of the IGL (F-IGL) prepared in E. coli is capable of providing the protective immunity for the liver abscess formation. In other words, since the IGL prepared in the E. coli was effective, modification by sugar chain may not always be necessary, and this indicates possibility of economically producing a large amount of IGL in E. coli system.

### Test Example 3

### Experiment of Immunization and Infection

Next, for the recombinant IGL which has been found to show the vaccine action, the region which may have the vaccine effect is identified in order to efficiently produce the fragment which can be produced at an efficiency higher than the full length IGL protein having a high molecular weight in E. coli system.

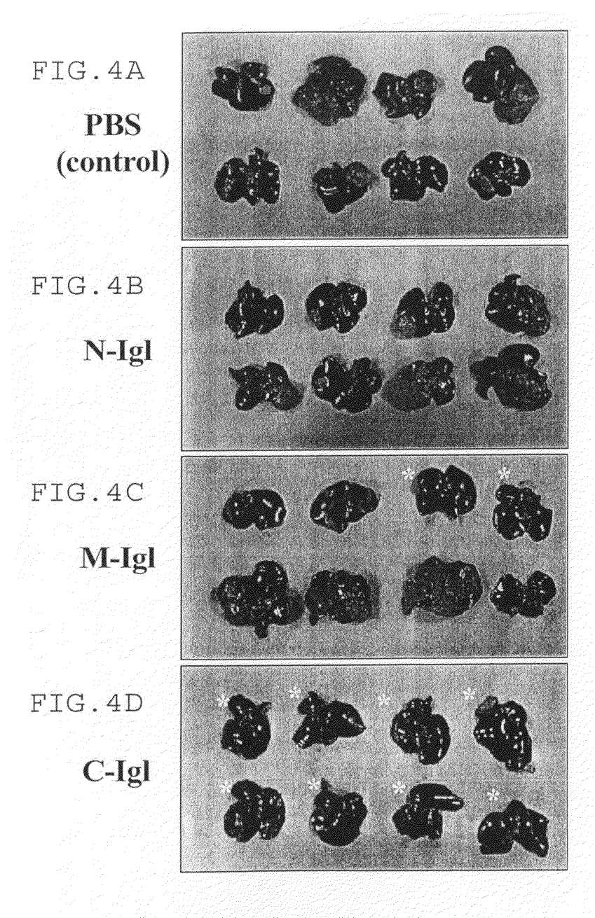

The experiment of inhibiting the formation of the amebic liver abscess by intramuscularly inoculating the recombinant IGL fragment of E. histolytica in hamster of Test Example 1 was repeated except that the F-IGL was replaced with the three fragments N-IGL, M-IGL, and C-IGL shown in Table 2. Thirty-two Syrian hamsters were used in this experiment. The results are shown in Table 5 and FIG. 4.

### [Table 5]

TABLE 5

| | | | Inhibition of | Average |
|---|---|---|---|---|
| | | Number of | the abscess | abscess |
| | Immunization | animals with | formation | size |
| Group | antigen | abscess | (%) | (wt %) |
| Control | PBS | 8/8 | 0 | 27 |
| 1 | N-IGL | 8/8 | 0 | 26 |
| 2 | M-IGL | 6/8 | 25 | 25 |
| 3 | C-IGL | 0/8* | 100 | — |

In relation to the control group, P = 0.000078

In FIG. 4, (A) is the control group inoculated with PBS, (B) is the group immunized with N-IGL, (C) is the group immunized with M-IGL, and (D) is the group immunized with C-IGL. In FIG. 4, * indicates that no abscess (indicated by an arrow) was found in the liver.

Of the 8 animals of the control group inoculated with the PBS, the abscess was found in all of the 8 animals. On the other hand, no animal with the abscess was found in the group immunized with C-IGL. The difference in Fisher's test is P=0.000078, which is a significant level. No preventive effect was found in the group immunized with N-IGL, and in the group immunized with M-IGL, significant difference with the control group was not found while no abscess was found in 2 animals out of the 8 animals. In addition, the group immunized with N-IGL or M-IGL exhibited no difference in the average abscess size with the PBS control group.

As evident from the results as described above, the antigen epitope(s) of the IGL which is capable of providing the preventive immunity for the liver abscess formation by E. histolytica in the experimental animal is included in the C-IGL fragment. C-IGL has a molecular weight of about 50,000, and the number of the amino acid residues (486) is less than half of the amino acid residue number of the full length IGL (1075), and therefore, efficient production in E. coli system is enabled.

As described above, immunization effect of the recombinant C-IGL was confirmed and possibility of its effective use in treating and preventing the amebic infection was indicated. Since the C-IGL prepared in E. coli was effective as described above, modification by the sugar chain may not always be necessary, and this indicated possibility for the mass production of amebiasis vaccine in the E. coli system at a reduced cost.

Sequence Listing

1. An isolated immunogenic E. histolytica protein fragment comprising an amino acid sequence including amino acid sequence 603-1088 of SEQ ID NO: 2.

2. A protein fragment of according to claim 1 comprising the amino acid sequence 603-1088 of SEQ ID NO: 2.

3. A protein fragment of according to claim 2 comprising the amino acid sequence 14-1088 of SEQ ID NO: 2.

4. An isolated DNA coding for the protein fragment of claim 1.

5. A vector including the DNA of claim 4.

6. A host cell containing the vector of claim 5.

7. A protein fragment according to claim 1 which is effective for amebiasis when used as a vaccine, wherein the effect is realized by the action that the antibody produced by administration of the protein fragment inhibits the infection process through cell adhesion or damages trophozoites through a complement- or ADCC-mediated action or by immunological mechanism such as cell-mediated immunity.

8. A protein fragment according to claim 1 wherein the protein fragment is free from sugar chains.

9. A protein fragment according to claim 1 wherein the protein fragment is a recombinant fragment produced by a prokaryote.

10. A method for producing an isolated immunogenic E. histolytica protein fragment comprising the steps of introducing the vector of claim 5 in a prokaryote to transform the host cell with the vector, cultivating the host cell, and collecting the resulting protein fragment.

11. A vaccine for amebiasis containing the protein fragment of claim 1.

12. A vaccine according to claim 11 wherein the protein fragment is a recombinant fragment produced by a prokaryote.

13. A vaccine according to claim 11 further comprising a component selected from the group consisting of adjuvant, excipient, and combinations thereof.

14. A method for inducing an immune response against E. histolytica in a mammal which is susceptible for infection by E. histolytica comprising the step of administering the protein fragment of claim 1 to the mammal as a component which exhibits immunogenicity in the host to provoke an immune response.

15. A method according to claim 14 wherein the mammal is human.

16. A method according to claim 14 wherein the protein fragment is a recombinant fragment produced by a prokaryote.

17. A method according to claim 14 wherein the protein fragment is administered with a component selected from the group consisting of adjuvant, excipient, and combinations thereof.

18. A method for preventing or treating infection by E. histolytica comprising the step of administering the protein fragment of claim 1 as an immunogenic component.

19. A method according to claim 18 wherein the protein fragment is a recombinant fragment produced by a prokaryote.

20. An antibody specifically binding to a protein fragment or a fragment thereof, wherein the protein fragment has an amino acid sequence containing an amino acid sequence selected from the group consisting of the amino acid sequence 603-1088 of SEQ ID NO: 2 and the amino acid sequence 14-1088 of SEQ ID NO: 2.


SEQUENCE LISTING (2 sequences)

SEQ ID NO: 1 - DNA, 3306 bp, Entamoeba histolytica
  CDS (1)..(3303)
atgtttattcttcttttattcatatcaatttcacttggtgattatactgctgataagctcataggcggaaaagagccaagagaggctgttccacattgtgcatcagtttcaaatggagcatgcactagttgtgatactggttatgaacttactaccactggaaataataagacatgtactcttaaagaagatatgtgtaaaactgctttttcttattatgataaaacaaactctacaaaccccaaatgtacttattgtgttaacggtaaagaagtaaatacatcatcacactctggaaatgataagtgtgtatgcaaaaacaatgtaaacatttgtgagtcatgtcttttgatgaaagattcaaaatgtggagaatgtataattgggatgtctactactgttgatggttcaaagttatgtgataatgcaactacagaagatcatgcagaaaattgtgttggtcttttagcctcttctacttcttcaaagacttgtgataaatgtttcggtatgtactctcttcaaggtgggaaatgtactcaaaagaatgataaaattaataaatgtattttacaagttgaaaactcttgtaaccaatgtgcagatggatattctctcagtactgataagaaatcttgtaataagttcccggagcattgctcaaagattaatggtaatcaatgcttgacatgtatggaaggttattatttaagtaaaacagattctaaatgtactatatgtactgttgataatccaaataatctttcagaaggtaacgaatgtagtatttataacgctgaacattgcacatcatgtaataaaagatgtactgtttctgatggagtttgtgtcaagaatcattgtcgtttattctcaccaacagaagaaaataaatgtacaaaatgtgataatggatatttcttaacaacttcaggaacatgttcaccaaatttgtatgatggtttcaaaacagctaatagaacagaatgtgaaaatggctattatttagaaaaagatggtgataaaaagagatgttcactttgtccagatccatttactgaatgtcttacttctaaaacaccagttccaggtaagttaaatcttagaagctcacacttaacatcaactgatggaccatgcaaacttccaggatgtttattatgtagtgatgatgatactatttgttataaatgtgagaatggacttacattgaatggaactcattgctataattttgacactaaatcagtccttggtactagtggtaacaaccatcaagtgtgtaagatgagaggatatgatcaatatgaacaatatttgaatgcatttaaagcatctgataatacttattattgtccacttaaagacctttatttaccatattatttcagtgttactaaaggtacttcagataatacaattactattggttgtgttggtcaattaagaaatgtttcaaatgactgtgaatgtaatgacaaacatattccaacatcaattgacaaagcatcagattgtgtttcaataacaaccaaacttccatcatgtgaaagaacagcaaatggaaatatttgtacacaatgtccagttggatcacatgtaggaaaggatggtaaatgttcttgtggtgatgcacattattttgacaaagataatgtctgtaaaaagtgtccagctagttgttcaagttgctcttatgatagttctaaaagtaaagttgtgtgtagtgaatgttatgaaaatattcaaggtgttactacaagaaataaagaaaatgaatgcgcttgcataaatgatggttataaagaaggaccaaatgcagaagataagaagaaaagttgtgcacaactaaataataattgtaaaaaggaaggtaaatatgaaattagtgatggatttgttacctgtcttgactgtgatgactcagcttatattgttggttcacaggttggtgcttgtacgcaatgttctcctaatgcttttaaagatgaaaataataaatgccaactttgttctactaaacaatctcaatatggacattgtgcagcatgttcagcaacagcatgtattacctgtgaagacattaacttaatacttaccggagaaaagccatgtactgtatgtaaagatggattttatcaaattgaaaatgcaacagatggagtgtattgtagtccatgtcctgcaaaatgtaaaacatgtaaatataataccacttcaaagaaagttgaatgtgtgacatgcactgaacaaaggctaaaagatattaaagcaccagaatgtgcttgtccaacaggaacagttcaacttgaaaatggaacgtgtcaaagttgctctgacctttcaaaatatccaggatgtaaaaaaactgattcatgtaatgttgatagtagaacaggatttatctatgcaacagaatgttcagatggttttagtggacgtagtccttatagtaattgtactacatgtactaagtctaattattatccaaaagaaggagaaaagaatgggtgtgctaaatgtgatgataaatgtgcaacatgttcagataaagacacttgtttaacatgtgctgatccattaaaggtaggaagtaaatgtgatggatgtaaaacaggttattatatgtcaaatggtgaatgtaagccatgtaccaatcattgtagtgaatgtagtagtgccgcagaatgtacagtatgtgagagtgatacatacaaggtaattagtggaaatggatgtaattcatgtgtagatggattctattttgatgagattaaaggaacatgtataccatgcacatcaccatgcactaaatgtgttggagtaaagaaagattgtgaagaacaagaaacaggatgtaattctgagaagaagaaaatagtagaagaatgtactaaatgttcaacaaaagatcatattgctgaagtaccagtgaatggagcatgtgtttgtgcatatggatatgttgaaggcacttctacagaagataataaaattgaatgtcaagcatgcaaagctaaagttaatgagttttgtgattcatgtaattccaaagactgtttgagatgcaatgctgaatatttagaagcaaaaggaggagaatgtgtatgtgttgaaggatattacacatctagttggggatcatgtattccatgttcaagacatatgccacattgtactaaatgtacaggagaaggcgaatgcacaacatgtgaagatggatggaaattaaaagatggaaagtgtaatggagctaaaggcattttcatcatgatgatgatagtcatgttagcatttatgttctaa

SEQ ID NO: 2 - protein, 1101 aa, Entamoeba histolytica
MFILLLFISISLGDYTADKLIGGKEPREAVPHCASVSNGACTSCDTGYELTTTGNNKTCTLKEDMCKTAFSYYDKTNSTNPKCTYCVNGKEVNTSSHSGNDKCVCKNNVNICESCLLMKDSKCGECIIGMSTTVDGSKLCDNATTEDHAENCVGLLASSTSSKTCDKCFGMYSLQGGKCTQKNDKINKCILQVENSCNQCADGYSLSTDKKSCNKFPEHCSKINGNQCLTCMEGYYLSKTDSKCTICTVDNPNNLSEGNECSIYNAEHCTSCNKRCTVSDGVCVKNHCRLFSPTEENKCTKCDNGYFLTTSGTCSPNLYDGFKTANRTECENGYYLEKDGDKKRCSLCPDPFTECLTSKTPVPGKLNLRSSHLTSTDGPCKLPGCLLCSDDDTICYKCENGLTLNGTHCYNFDTKSVLGTSGNNHQVCKMRGYDQYEQYLNAFKASDNTYYCPLKDLYLPYYFSVTKGTSDNTITIGCVGQLRNVSNDCECNDKHIPTSIDKASDCVSITTKLPSCERTANGNICTQCPVGSHVGKDGKCSCGDAHYFDKDNVCKKCPASCSSCSYDSSKSKVVCSECYENIQGVTTRNKENECACINDGYKEGPNAEDKKKSCAQLNNNCKKEGKYEISDGFVTCLDCDDSAYIVGSQVGACTQCSPNAFKDENNKCQLCSTKQSQYGHCAACSATACITCEDINLILTGEKPCTVCKDGFYQIENATDGVYCSPCPAKCKTCKYNTTSKKVECVTCTEQRLKDIKAPECACPTGTVQLENGTCQSCSDLSKYPGCKKTDSCNVDSRTGFIYATECSDGFSGRSPYSNCTTCTKSNYYPKEGEKNGCAKCDDKCATCSDKDTCLTCADPLKVGSKCDGCKTGYYMSNGECKPCTNHCSECSSAAECTVCESDTYKVISGNGCNSCVDGFYFDEIKGTCIPCTSPCTKCVGVKKDCEEQETGCNSEKKKIVEECTKCSTKDHIAEVPVNGACVCAYGYVEGTSTEDNKIECQACKAKVNEFCDSCNSKDCLRCNAEYLEAKGGECVCVEGYYTSSWGSCIPCSRHMPHCTKCTGEGECTTCEDGWKLKDGKCNGAKGIFIMMMIVMLAFMF